(19) United States
(12) Patent Application Publication
(10) Pub. No.: US 2005/0080332 A1
(43) Pub. Date: Apr. 14, 2005

(54) Near simultaneous computed tomography image-guided stereotactic radiotherapy

(76) Inventors: Almon S. Shiu, Bellaire, TX (US); Krishnan Ramaswamy, Burlington, MA (US); Zachary H. Leber, Cambridge, MA (US)

Correspondence Address: Mark Farber, Esq.;U.S. Surgical,, A Division of Tyco Healthcare Group, LP, 150 Glover Avenue, Norwalk, CT, 06856, US

(21) Appl. No.: 10/957,160

(22) Filed: Oct. 1, 2004

Related U.S. Application Data
(60) Provisional application No. 60/510,451, filed on Oct. 10, 2003.

(51) Int. Cl. A61B 5/00

(52) U.S. Cl. 600/411; 600/426; 600/427; 600/429; 600/414; 600/417; 606/130

(57) ABSTRACT

A targeting system for administering radiation to a patient and methods therefor are provided. The targeting system includes a stereotactic frame system for immobilizing the patient; an imaging scanner for acquiring images of a patient's anatomy, wherein at least one image is acquired during a planning phase, and at least one image is acquired during a pretreatment phase; a processor for fusing the planning image to the pretreatment image and for determining a shift, for example, translation and rotation, between the images to locate a predetermined portion of the patient's anatomy; and a radiation source for delivery of radiation to the predetermined portion of the patient's anatomy.

### PRIORITY

This application claims priority to an application entitled “NEAR SIMULTANEOUS COMPUTED TOMOGRAPHY IMAGE-GUIDED STEREOTACTIC SPINAL RADIOTHERAPY” filed in the United States Patent and Trademark Office on Oct. 10, 2003 and assigned Ser. No. 60/510,451, the contents of which are hereby incorporated by reference.

### BACKGROUND

1. Technical Field

The present disclosure is directed to an apparatus and methods for controlling the administration of radiation to a patient, and, in particular, to systems using near-simultaneous computed tomography (CT) image-guided stereotactic radiotherapy for the treatment of tumors and methods thereof.

2. Description of the Related Art

Of the 500,000 cancer-related deaths that occur annually, 40% involve patients with spinal metastases making the spine the most common site for bone metastasis. Although conventional radiation therapy can palliate pain and neurologic sequelae related to spinal metastases in 60-70% of cases, these metastases may progress or recur due to insufficient doses used because of dose constraints imposed by spinal cord tolerance. Furthermore, re-irradiation of the spine is rarely possible using conventional techniques.

Intracranial stereotactic radiosurgery has gained acceptance as a procedure for patients with limited brain metastases that has advantages over whole-brain irradiation in terms of conferring improved survival and tumor control, permitting re-irradiation, and sparing normal brain tissue. There is significant interest in implementing extracranial stereotactic radiotherapy. The implementation and delivery of extracranial stereotaxy, however, can pose formidable challenges involving immobilization and verification of the patient treatment position in three-dimensional (3D) space.

Stereotactic spinal radiotherapy or radiosurgery can play a complementary role to surgery in the management of spinal tumors. However, the success of this procedure largely depends on the accurate delivery of a highly conformal dose to a planning target volume and on sparing the spinal cord and other surrounding critical structures from radiation damage. A previously reported spinal radiosurgery technique involved the use of an invasive skeletal-fixation procedure to immobilize the vertebral bodies, see Hamilton A J, LuLu B A, “A prototype device for linear accelerator-based extracranial radiosurgery,” Acta Neurochir 1995;63:40-43. Small clamps were applied to the spinous processes to yield a placement error of 2 mm in the axial plane and <4 mm longitudinally. Not only was the procedure invasive, but also, because the patient was in the prone position, the setup accuracy was compromised by the vertebral body motion associated with patient breathing.

Several spinal procedures reportedly avoid the invasive approach of spinal irradiation. The CyberKnife™, commercially available from Accuray, Inc., of Sunnyvale, Calif., uses a robotic arm to deliver radiation in a wide range of beam orientations, except in the posterior region of the patient where two amorphous silicon (aSi) flat panel digital detectors are located. The CyberKnife™ relies on co-registration of digitally reconstructed radiographs (DRRs) generated from computed tomography (CT) images and x-ray projections captured before each node, e.g., a tumor, is treated with irradiation. Matching the mutual information from both data sets assumes that the treatment-planning geometry is reproduced at the time of each node treatment. Fiducial markers were implanted in the patient so that the treatment target could be determined based on the markers in the 2D x-ray images. However, co-registration of DRRs with the 2D x-ray images to achieve the submillimeter setup accuracy does not fully account for all types of motion because rotational errors are difficult to detect. In a study by Yin et al(see, Yin F, Ryu S, Ajlouni M, et al., “A technique of intensity-modulated radiosurgery (IMRS) for spinal tumors,” Med Phys 2002; 29:2815-2822.), who used a spinal radiosurgery technique that was a simplified version of the CyberKnife approach, the isocenter setup accuracy was within 2 mm.

A stereotactic paraspinal treatment that relies entirely on daily CT guidance was reported in Yenice K M, Lovelock D M, Hunt M A, et al., “CT image-guided intensity-modulated therapy for paraspinal tumors using stereotactic immobilization,” Int J Radiat Oncol Biol Phys 2003;55:583-593. This technique acquires planning CT images of 3-mm slice thickness and pretreatment CT images of 2-mm slice thickness. The position of the target and other anatomic landmarks are manually identified on the closely matched CT slices from both scans, and then their coordinates are computed in their frame's independent coordinate system. The new coordinates of all landmarks are compared with those determined from the planning CT study, and a daily setup deviation is assessed. According to this technique, a new isocenter position is determined on the basis of the average differences for all the landmarks used in the evaluation. The accuracy of reproducing the treatment-planning geometry heavily depends on the accuracy of manually identifying the same anatomic landmarks and the position of the target. The patient, positioned inside a stereotactic body frame (SBF), is then transferred to the treatment table using a rail system. Rotation around yaw can occur when the SBF is transferred from the CT couch to the treatment table. The pitch may also vary owing to the different structures of the CT couch and the treatment table. Neither yaw nor pitch was addressed. Patient position is verified by comparing DRRs with the portal images before the treatment is delivered.

### SUMMARY

A targeting system for administering radiation to a patient and methods therefor are provided. The targeting system integrates a CT scanner with a linear accelerator (LINAC) for treating patients with paraspinal metastases, cranial tumors, etc. During the CT image-guided stereotactic radiotherapy procedure, a patient is immobilized to minimize body motion. During a planning phase, images of the patient's anatomy are obtained, e.g., planning CTs, and subsequent images are obtained prior to each treatment, e.g., pretreatment CTs. The pretreatment CT images provide a solid link between the patient anatomy and the stereotactic coordinate system. By fusing the daily pretreatment CT images with the planning CT images, the technique of the present disclosure can determine how the patient and especially the body target has shifted, rotated, or both with respect to the planning isocenter. The movement is then compensated to deliver a highly accurate dose of radiation.

According to an aspect of the present disclosure, a targeting system for administering radiation to a patient is provided. The system includes a stereotactic frame system for immobilizing the patient; an imaging scanner for acquiring images of a patient's anatomy, wherein at least one image is acquired during a planning phase, and at least one image is acquired during a pretreatment phase; a processor for fusing the planning image to the pretreatment image and for determining a shift between the images to locate a predetermined portion of the patient's anatomy; and a radiation source for delivery of radiation to the predetermined portion of the patient's anatomy.

In another aspect, the stereotactic frame system is a body frame including a base plate for supporting the patient; a whole body vacuum cushion and fixation sheet for securing the patient in a predetermined position; and, a vacuum for removing air from the cushion and between the cushion and fixation sheet to fix the patient in the predetermined position. The stereotactic body frame system further includes a stereotactic localizer for determining a position of the patient's anatomy relative to the radiation source and a target positioning frame for positioning the radiation source to deliver radiation to the predetermined portion of the patient's anatomy.

According to a further aspect of the present disclosure, a method for administering radiation to a patient is provided. The method includes the steps of obtaining a first image of a predetermined portion of the patient's anatomy during a planning phase of a treatment procedure; obtaining a second image of the predetermined portion of the patient's anatomy during a pretreatment phase of the treatment procedure; fusing the first image to the second image to align the first and second images; determining a shift to align the first and second images; calculating an isocenter of the patient's anatomy in the second image using the first image and the determined shift.

In a further aspect of the present disclosure, a program storage device readable by machine, tangibly embodying a program of instructions executable by the machine to perform method steps for determining a target volume in a patient is provided, wherein the method steps include obtaining a first image of a predetermined portion of the patient's anatomy during a planning phase of a treatment procedure; obtaining a second image of the predetermined portion of the patient's anatomy during a pretreatment phase of the treatment procedure; fusing the first image to the second image to align the first and second images; determining a shift to align the first and second images; and calculating an isocenter of the target volume of the patient in the second image using the first image and the determined shift.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1 illustrates an exemplary targeting system for administering radiation to a patient according to an embodiment of the present disclosure;

FIG. 2 is a flow chart illustrating a method for administering radiation to a patient according to an embodiment of the present disclosure;

FIGS. 3A and 3B illustrate a method for immobilizing a patient;

FIG. 4 is a flow chart illustrating a method for fusing planning images to pretreatment images to determine patient shift;

FIG. 5 illustrates an exemplary targeting system of the present disclosure delivering radiation to a target's isocenter;

FIG. 6 illustrates superimposed axial and sagittal images from reference CT scans and immediately repeated CT scans using alignment of localization rods on the stereotactic body frame;

FIG. 7 illustrates an example of a phantom study to illustrate the linkage between patient anatomy and the stereotactic localization frame;

FIG. 8(a) illustrates seven coplanar IMRT (Inverse Planning Intensity-Modulated Radiotherapy) beams relative to patient anatomy in CT images, FIGS. 8(b) and (c) illustrate isodose distributions in axial and sagittal views, respectively, and FIG. 8(d) illustrates cumulative dose volume histograms for CTV (clinical target volume), spinal cord, and the other adjacent critical structures; and

FIG. 9(a) illustrates the uncorrected daily setup deviation from the planned isocenter in each of the AP (anterior-posterior), LAT (lateral), and SI (superior-inferior) directions over 15 treatments for three patients, FIG. 9(b) illustrates the deviation of corrected daily isocenter from the planning isocenter in each of the three directions over the 15 treatments for three patients and FIG. 9(c) illustrates the directions of positive deviations corresponded to a shift of the planning target isocenter with respect to the patient in the right, anterior, and superior directions.

### DETAILED DESCRIPTION

Embodiments of the present disclosure will be described herein below with reference to the accompanying drawings. In the following description, well-known functions or constructions are not described in detail to avoid obscuring the disclosure in unnecessary detail. In the figures, like reference numerals represent like elements.

A near simultaneous computed tomography (CT) image-guided stereotactic radiotherapy apparatus and technique are provided. A patient is immobilized in a stereotactic body frame system to minimize intra-treatment movement and vertebral body motion associated with breathing. The use of pretreatment daily CT scans in conjunction with planning CT scans enabled the apparatus to accurately target a tumor regardless of daily setup variations in patient position within the immobilization device. Although the following description describes a CT-on-rails system with a movable couch and stereotactic localizer, the present disclosure contemplates other systems which employ scanning means and radiation delivery means such as a cone beam CT system.

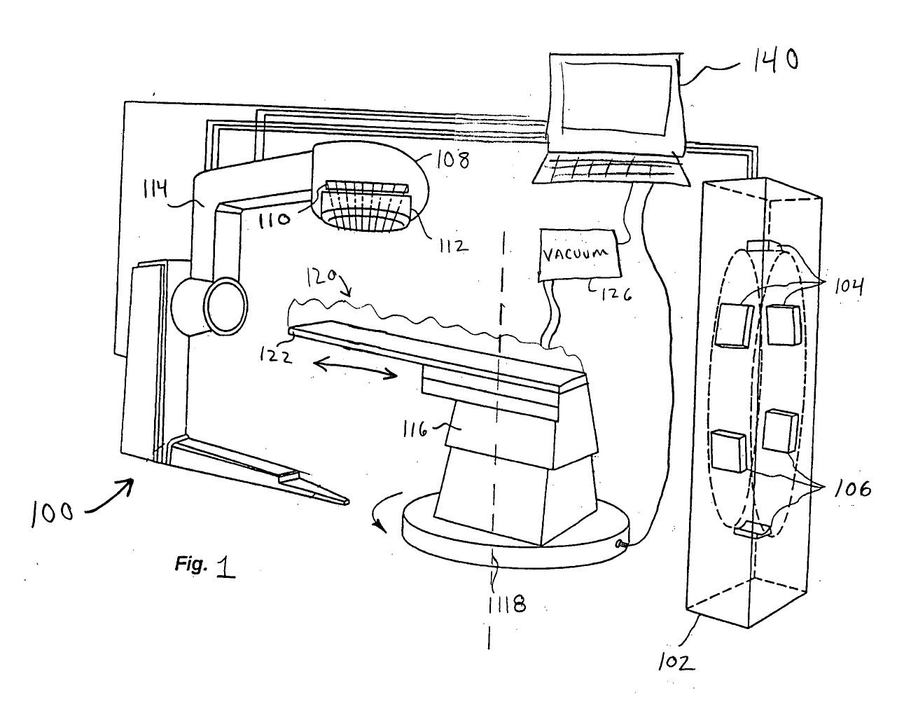

Turning in detail to the drawings, FIG. 1 illustrates a targeting system 100 for administering radiation to a patient according to an embodiment of the present disclosure. The targeting system includes an image scanner 102 for acquiring images of a patient's anatomy such as a CT, MRI unit or other patient visualization means containing X-ray tubes or comparable means 104 and detectors 106. The targeting system further includes a radiation source beam assembly (RSBA) 108 containing a radiation source beam assembly unit (RSBU) 110, which contains radioactive sources which emit beams of radiation, intersecting to form a treatment zone. The RSBA 108 includes a collimator 112. The RSBA 108 is supported on a rotatable source support means such as gantry 114. A treatment table 116 for supporting a patient is also included which is rotatable about a central axis 118 and movable in a horizontal direction, preferably on rails, to move a patient from a scanning position in close proximity to the image scanner 102 to a treatment position in close proximity to the RSBA 108. An exemplary targeting system is commercially embodied in the EXaCT™ targeting system, from Varian Medical Systems of Palo Alto, Calif., which integrates a CT scanner with a linear accelerator (LINAC) unit. This system is equipped with a high-speed CT scanner on rails, from GE Medical Systems of Milwaukee, Wis., and a linear accelerator with Millennium 120 multileaf collimator (MLC) as well as dual photon energies of 6 MV and 18 MV. Sixty pairs of leaves (40 pairs with a leaf width of 5 mm and 20 pairs with leaf width of 10 mm) form a maximum field size of 40×40 cm. The advantage of this integrated system is that the treatment table 116 can be swung to position the patient for either treatment or scanning, without having to move the patient from the treatment table to a CT couch.

The evaluation of mechanical precision and alignment uncertainties for the EXaCT™ integrated CT/LINAC system was reported in Court, L. et al., “Evaluation of mechanical precision and alignment uncertainties for an integrated CT/LINAC system” Med Phys 30:1-13, 2003. The following sources of uncertainties were identified: (1) the patient couch position on the LINAC side after a rotation, (2) the patient couch position on the CT side after a rotation, (3) the patient couch position as indicated by the digital readout, (4) the difference in couch sag between the CT and LINAC positions, (5) the precision of the CT coordinates, (6) the identification of fiducial markers from CT images, (7) the alignment of contours with structures in the CT images, and (8) the alignment with setup lasers. The largest single uncertainty (one standard deviation or 1 SD) was found in the couch position on the CT side after a rotation (0.5 mm in the LAT direction) and the alignment of contours with the CT images (0.4 mm in the SI direction). All other sources of uncertainty were less than 0.3 mm (1 SD).

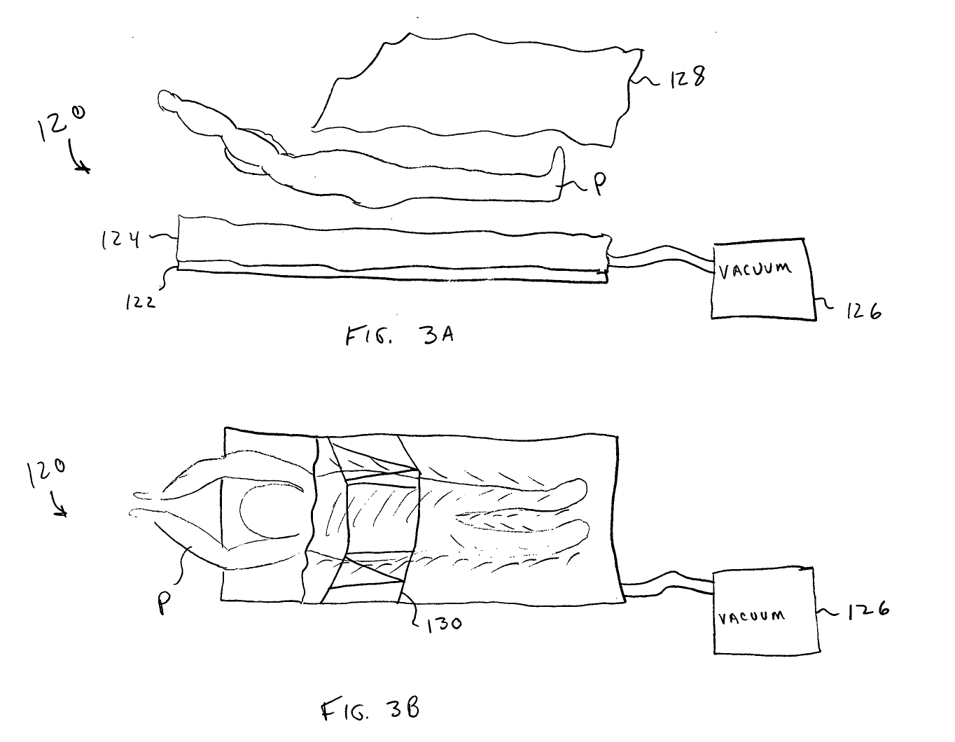

61 To overcome these uncertainties, the targeting system 100 of the present disclosure further includes a stereotactic body frame system (SBFS) 120 for immobilizing the patient. Referring to FIGS. 1 and 3, the SBFS 120 includes of a carbon fiber base plate 122 for supporting the patient and a whole-body vacuum cushion 124, vacuum system 126 and plastic fixation sheet 128 for fixing the patient to a stationary position. The SBFS 120 further includes a stereotactic localizer 130 for determining a position of the patient's anatomy relative to the RSBA 108 and a target positioning frame for positioning the RSBA 108 to deliver radiation to an isocenter of a target in the patient. Optionally, a rigid structure, e.g., Lucite blocks, will preserve the shape of the vacuum cushion on its lateral aspects and an arm support system will support the arms of the patient above the patient's head.

Although the present disclosure describes utilizing a stereotactic body frame to immobilize the patient during a spinal treatment, it is to be appreciated that other devices may be employed to immobilize a target of interest of a patient. For example, when treating a cranial tumor, a head frame will be employed.

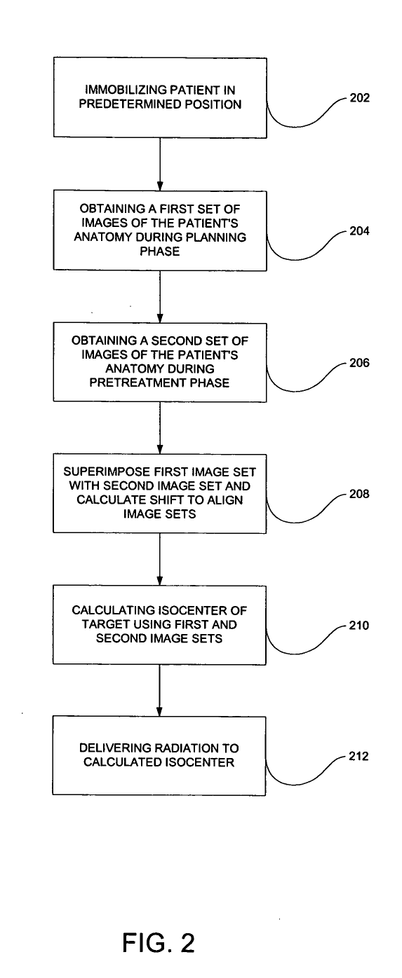

A computer 140 is coupled to the image scanner 102, RSBA 108, and the treatment table 116 for controlling the overall operations of the targeting system which will be described below in relation to FIG. 2. Preferably, the computer is implemented on a computer platform having hardware such as one or more central processing units (CPU), a random access memory (RAM), and input/output (I/O) interface(s) such as a keyboard, cursor control device (e.g., a mouse) and display device. The computer platform also includes an operating system and micro instruction code. The various processes and functions described herein may either be part of the micro instruction code or part of an application program (or a combination thereof) which is executed via the operating system. In addition, various other peripheral devices may be connected to the computer platform such as an additional data storage device and a printing device. It is to be understood that the present disclosure may be implemented in various forms of hardware, software, firmware, special purpose processors, or a combination thereof.

Referring to FIG. 2, a method for administering radiation to a patient according to the present disclosure will now be described. It is to be further understood that, because some of the constituent system components and method steps depicted in the accompanying figures may be implemented in software, the actual connections between the system components (or the process steps) may differ depending upon the manner in which the present disclosure is programmed. In one embodiment, the present disclosure may be implemented in software as an application program tangibly embodied on a program storage device. The application program may be uploaded to, and executed by, a machine comprising any suitable architecture. Given the teachings of the present disclosure provided herein, one of ordinary skill in the related art will be able to contemplate these and similar implementations or configurations of the present disclosure.

Initially, in step 202, the patient is immobilized in a predetermined position. Referring to FIGS. 3A and 3B the patient P lies down in the supine position in the body cushion 124 supported by the carbon fiber base plate 122. The body cushion 124 is then custom-molded to the shape of the patient's body by removing the air from the cushion via vacuum system 126. Fluoroscopic imaging, performed on a conventional simulator, is then employed to ensure the patient is lying straight and flat on the patient's back. A plastic fixation sheet 128 is then placed on top of the patient and sealed on the sides of the body cushion. The patient's arms are raised over the patient's head to rest on the arm-supporting device (not shown). A vacuum pump and hose attachment of the vacuum system 126 then removes all the air surrounding and between the vacuum cushion 124 and the fixation sheet 128, essentially “shrink wrapping” the patient and conforming the fixation sheet to the actual shape of the patient's body.

Once the patient is immobilized, a set of images are acquired by the image scanner 102 during a planning phase with the stereotactic localizer 130 attached to the SBFS 120 (step 204). The planning images, or planning CTs, are obtained with a pixel resolution of 0.98 mm×0.98 mm and a slice thickness of 2 mm. The number of slices for a typical planning scan ranges from 90 to 100 slices, the acquisition time is approximately 2 minutes. During the image acquisition, the custom body cushion and the localizer 130 were locked onto an index position of the treatment table 116.

A major drawback of conventional noninvasive SBFSs is the lack of a robust and direct spatial link between patient anatomy and the localization frame or localizer. Despite the best effort to immobilize the patient using the conventional SBFS, it is still difficult to precisely reposition the patient for daily paraspinal treatments that require setup accuracy to be within 2 mm. By performing pretreatment CT imaging immediately before each treatment, a robust link is created between the patient's anatomy and the SBFS.

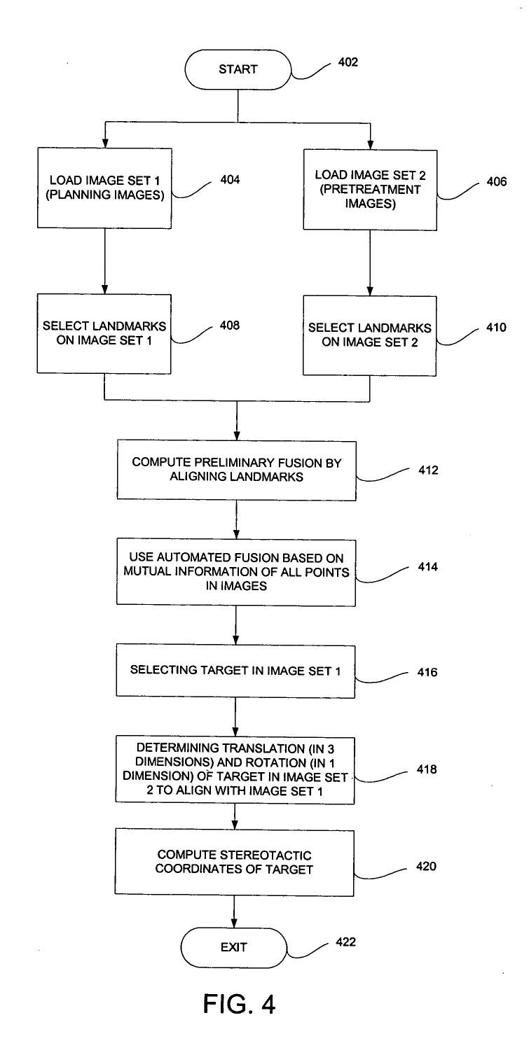

Before each treatment, the patient is set up in the stereotactic body immobilization system, as previously described. Next, a pretreatment CT is obtained using the same slice thickness of 2 mm as with the planning CT (step 206). The planning CT images are then superimposed with the pretreatment CT images using automated fusion software restricted to spinal bony anatomy only (step 208), as will be described in more detail in relation to FIG. 4.

In one embodiment, after the image scanner 102 has obtained the planning and pretreatment CTs, the fusion software is initiated in computer 120 (step 402). The planning images are loaded and possibly displayed on a display of the computer (step 404) and the same is performed for the pretreatment CTs (step 406). A plurality of landmarks is then selected on the planning CT (step 408). At least three landmarks should be selected to ensure proper registration of the images. Selecting of the landmarks may be done manually via a mouse while visually observing the planning CT on the display or the selection may be made automatically by image detection and segmentation software as is known in the art. The same landmarks are then selected on the pretreatment CT (step 410) although they may be in a different position due the patient shifting from the planning phase to the pretreatment phase. The selected landmarks are then aligned by translating, e.g. in three dimensions, and rotating, e.g., in one dimension, the second image (step 412). This preliminary fusion of images is based solely on the landmarks selected.

Next, an automatic fusion process fuses the two images together based on all points, e.g., voxels, in the images (step 414). The automatic fusion software employed is based on mutual information (MI), as described in “Medical Image Registration Using Mutual Information”, Maes et al., Proceedings of the IEEE, Vol. 91, NO. 10, Oct. 2003, the contents of which are incorporated by reference. Mutual information is a basic concept from information theory, measuring the statistical dependence between two random variables or the amount of information that one variable contains about the other. The MI registration criterion states that the mutual information of the image intensity values of corresponding voxel pairs is maximal if the images are geometrically aligned. This automatic fusion software allows for robust and completely automated registration of multimodal images without other processing steps, which makes this method very well suited for clinical applications.

Alternatively, the two images may be aligned through mutual information alone without selecting any landmarks—skipping steps 408 and 410.

Once the planning CT is registered with the pretreatment CT, the isocenter point for the target of the patient's current position can be calculated. The target is selected on the pretreatment CT (step 416) and then the shift and the axial rotation of the isocenter point from the planning CT to the pretreatment CT are calculated (step 418) based on the registration matrix and displayed onscreen. Once the location of the isocenter point in the pretreatment CT is known, the final step is to compute the stereotactic coordinates of that point using the stereotactic localizer affixed to the patient during the pretreatment CT (step 420). The pretreatment stereotactic coordinates are compared to the planning stereotactic coordinates to determine the final shift.

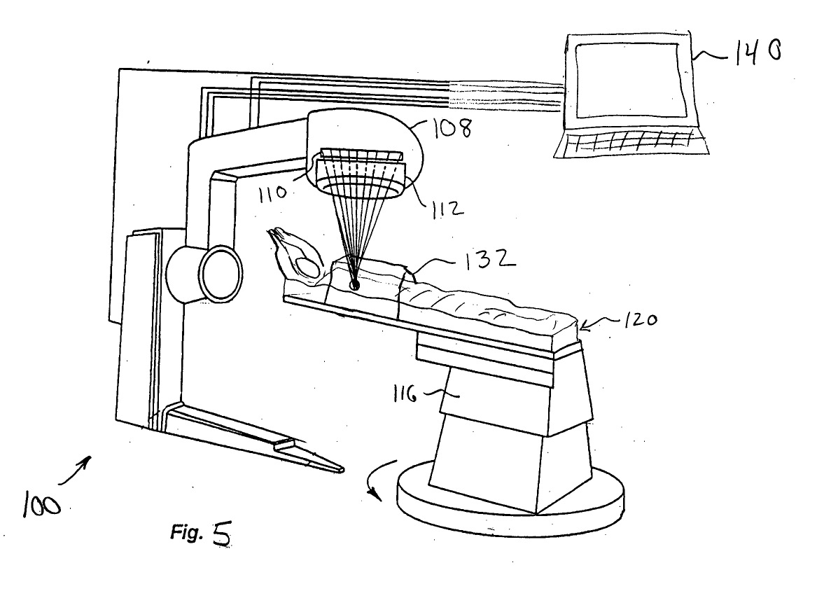

The shift and rotation is then entered into a treatment-planning system to generate the corrected daily anterior-posterior (AP) and right lateral (RT LAT) DRRs for targeting the isocenter (step 210). Preferably, the treatment-planning system is an application software program running on the computer 140 of system 100. The corrected target isocenter is marked directly on overlay sheets, which were attached to a respective side of the target positioning frame 132, as shown in FIG. 5. The planning isocenter is indicated on the right (RT), left (LT), and anterior (ANT) overlay sheets, respectively. Each overlay sheet has stereotactic coordinates in millimeter scales. The scales of each overlay sheet are carefully aligned with the millimeter incremented scales on the plates of the target positioning frame. The separation of the pretreatment isocenter and the planning isocenter should corresponding to the shift calculated from the fusion software. The patient is then positioned for radiotherapy by rotating the treatment table 116 180°.

The target-positioning frame 132 is then placed in the same index position as that of the stereotactic localizer 130. The patient is set to the updated correct isocenter using the target-positioning frame and lasers. The positioning frame is removed, and prior to the delivery of radiation treatment, orthogonal portal images are acquired. If axial rotation was detected from the fusion calculation, the gantry angle for obtaining the AP and RT LAT portal film is corrected by adding or subtracting the angle of the roll. These orthogonal portal images are then compared with the planning DRRs for the final verification step. Once verified, radiation is delivered to the updated isocenter via the RSBA 108 (step 212).

To verify the accuracy of the above technique, various experiments were performed, the results of which are described below.

Treatment planning for fractional stereotactic radiotherapy was performed with inverse planning intensity-modulated radiotherapy (IMRT) software, e.g., P³IMRT commercially available from Pinnacle Elekta, of Norcross, Ga. The clinical target volume (CTV) and critical organs such as the spinal cord, kidneys, and lungs were contoured. The CTV plus a 3-mm margin constituted the planning target volume (PTV) and a margin of 3 mm was also added to the spinal cord. However, the expansion of the CTV was not extended into the critical organs. Coplanar IMRT using 7-9 beams delivered 30 Gy in 5 fractions to the PTV while limiting spinal cord dose to <10 Gy. After completion of the IMRT planning, the full set of IMRT fields defined for each patient was subjected to a quality assurance (QA) phantom image set, in which IC-04 ion-chamber (0.04 cm³ active volume, IBA-Scanditronix Wellhofer, Memphis, Tenn.) measurements at the isocenter of the high-dose region was carried out. In addition, an axial isodose distribution was measured with extended dose range (EDR) film. Both measurements were compared with the calculation generated by treatment-planning software.

Based on a total of 36 CT scans (three for planning, three for respiration study, 15 pretreatment, and 15 post-treatment) from three patients, no respiration-associated vertebral body motion was observed. A comparison of the corrected daily anterior-posterior (AP) and lateral (LAT) digital portal images with the planning AP and LAT DRRs confirmed that the isocenter setup accuracy for the 15 treatments was within 1 mm of the planning isocenter. The results from the immediate post-treatment CT scans reconfirmed the findings from the portal images and verified the absence of spinal movement during the treatment. The ion-chamber measurement for the high-dose region was within 2% of the planning dose for three patient treatment plans. Film dose measurement in an IMRT QA phantom demonstrated good agreement from 90% to 30% isodose lines between the planned and measured results.

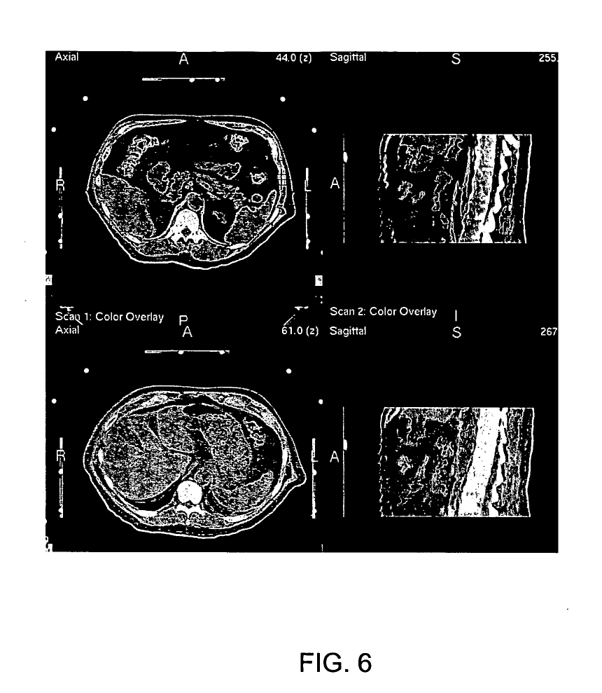

FIG. 6 shows the superimposed axial and sagittal images generated from the imaging fusion software using manual fusion to align rods of the localizer contained within the reference CT images and the immediately repeated CT images. Since the localization frame was not moved during the reference CT and the immediately repeated CT, the localization rods (labeled R and L) matched exactly. Under this condition, the movement of any anatomy associated with normal breathing, especially the vertebral bodies, could be quantified. All vertebral bodies perfectly overlap indicating that no vertebral body motion was observed, even during respiration, when the patient was properly immobilized in the body system. When a patient was lying in the SBFS without the use of plastic fixation sheets, however, the vertebral bodies moved substantially (>1 cm) in a superior-inferior (SI) direction during fluoroscopy.

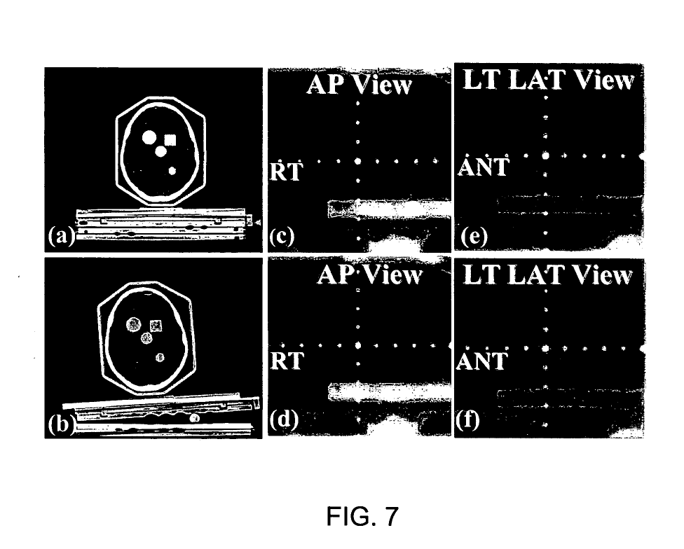

The ability to fuse the daily CT images with the planning CT images is a major strength of the technique of the present disclosure. By comparing pretreatment and planning CT images using fusion software, the correct target isocenter can be determined by accounting for any translational and rotational discrepancies compared with the planning CT. FIG. 7 demonstrates how to link the patient anatomy with the stereotactic localization frame.

FIG. 7(a) illustrates an axial CT scan obtained without applying any shift, representing the planning CT and FIG. 7(b) illustrates a daily CT scan, e.g., a pretreatment CT, shifted and rotated from the original planning position, indicating patient deviation from the original position. The center of the sphere was designed as the treatment isocenter. The corrected daily isocenter was updated on the basis of the fusion results. To aim toward the spherical target in the daily setup in a manner identical to that used in the planning setup situation, the planned isocenter needed to move laterally to the right 31.5 mm, vertically to the inferior 18 mm, and anteroposteriorly to the anterior 7 mm. Roll correction was made by rotating the gantry counterclockwise 3.9°. A comparison of AP and LT LAT views of portal images between the planning isocenter (FIGS. 7(c) and 7(e), respectively) and updated daily isocenter (FIGS. 7(d) and 7(f), respectively) confirms the setup accuracy to be within 1 mm. A comparison of the axial plan and daily CT images by image fusion can yield information that is necessary to perform corrections for translation, including AP, mediolateral, and SI, as well as rotation around the AP axis (yaw), rotation around the mediolateral axis (pitch), and rotation around the SI axis (roll).

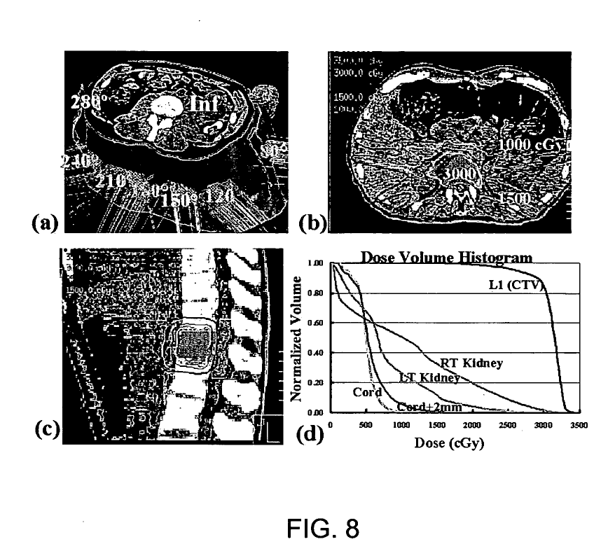

FIG. 8(a) illustrates seven coplanar IMRT beams relative to patient anatomy in CT images and the beam angles at 280°, 240°, 210°, 180°, 150°, 120°, and 80°. The beam energy was 6-MV photons, except for 18-MV photons used for the 280° and 80° beams, which traversed more tissue before striking the target. Isodose distributions for the axial and sagittal views are shown in FIG. 8(b) and 8(c), respectively. The isodose was normalized to the isocenter. Typically, the dose was prescribed to the volume included by the 90% isodose line. As shown in the figures, a small portion of the CTV received less than 30 Gy of the prescription dose. The ultimate prescription dose was constrained by the IRB requirement for protocol approval that the dose to the spinal cord could not exceed a total of 10 Gy over five treatments. Thus it was necessary that each treatment plan achieve an acceptable compromise between the target coverage and the cord dose limitation. The dose volume histogram (FIG. 8(d)) illustrates the accuracy of the targeting system of the present disclosure.

For each patient treated with IMRT, the absolute dose measurements for the isocenter using the IMRT QA water phantom indicated that the median deviation of the measured isocenter dose from the planning isocenter dose for the three patients was 1.7%, with a maximum deviation of 2.1%. In addition, film measurements in an IMRT QA phantom demonstrated good agreement from 90% to 30% isodose lines between the planned and measured results.

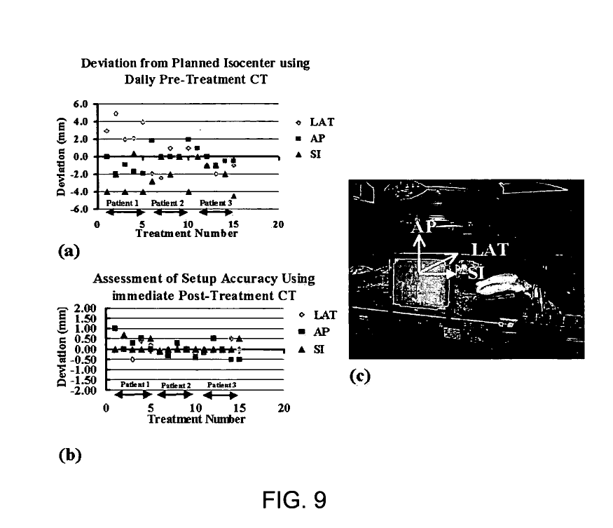

On the bases of the pretreatment CT scans, the daily setup deviation from the planned isocenter in each direction over the 15 treatments for three patients, as determined, is shown in FIG. 9(a). The deviation from the planned isocenter ranged from −2.4 mm to 4.9 mm in the LAT direction, from −1.95 mm to 1.8 mm in the AP direction, and from −4.5 mm to 0.4 mm in SI direction (mean deviation±standard deviation [± SD]: such as 0.59±2.21 mm in the LAT direction, −0.26±1.2 mm in the AP direction, and −2.06±1.74 mm in the SI direction).

Using the conventional stereotactic body system alone, it is not possible to achieve the setup accuracy necessary for delivering a highly conformal dose of radiation to the target. With almost real-time CT image guidance, in accordance with the present disclosure, the corrected daily isocenter is marked on the positioning frame. For final verification before delivery of the treatment, a pair of orthogonal portal films was taken and compared with the planning DRRs to confirm that the patient setup was correct. Based on the immediate post-treatment CT used to reveal any intra-treatment motion, FIG. 9(b) shows the daily setup deviation from the planned isocenter in each direction over the 15 treatments for three patients. Except for two data points, all the data indicated that the deviation from the planned isocenter in each of the three directions was within 0.5 mm (mean deviation±SD: 0.04±0.22 mm in the LAT direction, 0.03±0.42 mm in the AP direction, and 0.11±0.24 mm in the SI direction). The overall deviation from the planning isocenter for each treatment was within 1 mm. FIG. 9(c) shows that the directions of positive deviations corresponded to a shift of the planning target isocenter with respect to the patient in the right, anterior, and superior directions.

Paraspinal tumors can be accurately targeted by radiotherapy only if physical dose delivery, patient positioning, and target localization are extremely accurate. Using the nearly simultaneous CT image-guided stereotactic radiotherapy technique of the present disclosure, the maximum deviation of actual treatment isocenter from the planning isocenter in each of the LAT, AP, and Si directions is 1 mm.

The key distinguishing feature of the technique of the present disclosure is that it uses true stereotactic localization methods enhanced by near-simultaneous CT image-guidance and computerized imaging registration to tightly link patient anatomy to the SBFS. Excellent immobilization of the patient using the SBFS and stereotactic localization enhanced by near-simultaneous CT image-guidance and computerized image registration are critical to delivering highly accurate treatments. Using this technique, setup accuracy of within 1 mm was achieved.

While several embodiments of the disclosure have been shown in the drawings, it is not intended that the disclosure be limited thereto, as it is intended that the disclosures be as broad in scope as the art will allow and that the specification be read likewise. Therefore, the above description should not be construed as limiting, but merely as exemplifications of preferred embodiments.

1. A targeting system for administering radiation to a patient comprising:
a stereotactic frame system for immobilizing the patient;
an imaging scanner for acquiring images of a patient's anatomy, wherein at least one image is acquired during a planning phase, and at least one image is acquired during a pretreatment phase;
a processor for fusing the planning image to the pretreatment image and for determining a shift between the images to locate a predetermined portion of the patient's anatomy; and
a radiation source for delivery of radiation to the predetermined portion of the patient's anatomy.

2. The targeting system of claim 1, wherein the stereotactic frame system is a body frame system including:
a base plate for supporting the patient;
a whole body vacuum cushion and fixation sheet for securing the patient in a predetermined position; and
a vacuum for removing air from the cushion and between the cushion and fixation sheet to fix the patient in the predetermined position.

3. The targeting system of claim 1, wherein the stereotactic frame system includes a stereotactic localizer for determining a position of the patient's anatomy relative to the radiation source.

4. The targeting system of claim 1, wherein the stereotactic frame system includes a target positioning frame for positioning the radiation source to deliver radiation to the predetermined portion of the patient's anatomy.

5. The targeting system of claim 1, wherein the stereotactic frame system is rotatable about a first axis and slidable about a second axis.

6. The targeting system of claim 1, wherein the stereotactic frame system is movable from a scanning position to a treatment position while the patient is immobilized.

7. The targeting system of claim 1, wherein the radiation source is a linear accelerator.

8. The targeting system of claim 1, wherein the imaging scanner is a computed tomography (CT) scanner.

9. The targeting system of claim 1, wherein the imaging scanner is a magnetic resonance imaging (MRI) scanner.

10. A method for administering radiation to a patient, the method comprising the steps of:
obtaining a first image of a predetermined portion of the patient's anatomy during a planning phase of a treatment procedure;
obtaining a second image of the predetermined portion of the patient's anatomy during a pretreatment phase of the treatment procedure;
fusing the first image to the second image to align the first and second images;
determining a shift to align the first and second images; and
calculating an isocenter of the patient's anatomy in the second image using the first image and the determined shift.

11. The method of claim 10, further comprising the step of delivering radiation to the calculated isocenter.

12. The method of claim 10, wherein the determining a shift step includes calculating a translation of at least one of the first and second images in three dimensions.

13. The method of claim 10, wherein the determining a shift step includes calculating a rotation of at least one of the first and second images in one dimension.

14. The method of claim 10, wherein the fusing step includes selecting a plurality of landmarks in the first image and selecting identical landmarks in the second image and aligning the selected landmarks of the first and second images.

15. The method of claim 14, wherein the selecting of the plurality of landmarks step is performed by image detection software.

16. The method of claim 14, wherein the fusing step further includes fusing each voxel of the first image to each voxel of the second image after the selected landmarks are aligned.

17. The method of claim 10, further comprising the steps of:
determining stereotactic coordinates of the isocenter in relation to a immobilization device affixed to the patient.

18. A program storage device readable by machine, tangibly embodying a program of instructions executable by the machine to perform method steps for determining a target volume in a patient, the method steps comprising:
obtaining a first image of a predetermined portion of the patient's anatomy during a planning phase of a treatment procedure;
obtaining a second image of the predetermined portion of the patient's anatomy during a pretreatment phase of the treatment procedure;
fusing the first image to the second image to align the first and second images;
determining a shift to align the first and second images; and
calculating an isocenter of the target volume of the patient in the second image using the first image and the determined shift.

19. The program storage device of claim 18, wherein the determining a shift step includes calculating a translation of at least one of the first and second images in three dimensions.

20. The program storage device of claim 18, wherein the determining a shift step includes calculating a rotation of at least one of the first and second images in one dimension.

21. The program storage device of claim 18, wherein the fusing step includes selecting a plurality of landmarks in the first image and selecting identical landmarks in the second image and aligning the selected landmarks of the first and second images.

22. The program storage device of claim 21, wherein the selecting of the plurality of landmarks step is performed by image detection software.

23. The program storage device of claim 21, wherein the fusing step further includes fusing each voxel of the first image to each voxel of the second image after the selected landmarks are aligned.

24. The program storage device of claim 18, further comprising the step determining stereotactic coordinates of the isocenter in relative to a fixed point of the patient.
